(19) United States
(12) Patent Application Publication
(10) Pub. No.: US 2020/0261614 A1
(43) Pub. Date: Aug. 20, 2020

(54) VASCULAR GRAFT AND METHODS FOR SEALING A VASCULAR GRAFT

(71) Applicant: TC1 LLC, St. Paul, MN (US)

(72) Inventor: Ramesh Babu Jayaraman, Fremont, CA (US)

(21) Appl. No.: 16/793,470

(22) Filed: Feb. 18, 2020

Related U.S. Application Data
(60) Provisional application No. 62/807,498, filed on Feb. 19, 2019.

(51) Int. Cl. A61L 24/00; A61L 24/04

(52) U.S. Cl. CPC A61L 24/0031; A61M 1/1008; A61L 24/043

(57) ABSTRACT

Disclosed herein are a vascular graft and a method for sealing a vascular graft. The method generally includes applying a hydrogel sealant to the vascular graft thereby sealing the graft, wherein the hydrogel sealant is prepared from material selected from the group consisting of plant sourced material, synthetic material, or a combination thereof. In some embodiments, the hydrogel sealant may be prepared from albumin and a crosslinking agent or from poly(ethylene)oxide and a poly(amine).

### CROSS-REFERENCE TO RELATED APPLICATIONS

This application claims priority to U.S. Provisional Patent Application Ser. No. 62/807,498, filed on Feb. 19, 2019, the disclosure of which is hereby incorporated herein by reference in its entirety.

### FIELD OF THE DISCLOSURE

The present disclosure relates generally to medical devices that are used in the human body. In particular, the present disclosure relates to various vascular grafts and methods for sealing vascular grafts using flexible and biocompatible hydrogel sealants.

### BACKGROUND

Vascular grafts are commonly used in combination with medical devices such as ventricular assist devices. Many vascular grafts are constructed from large diameter knitted or woven polyester materials that perform well within the human body. In some cases, these large-diameter knitted or woven polyester materials may leak blood for a short period of time after introduction into the body due to the high porosity of the graft material. In many cases, to minimize or eliminate blood leakage through the grafts, the grafts may be sealed with one or more materials. In some particular cases, the grafts may be sealed with gelatin derived from a bovine source (e.g., VASCUTEK® Grafts). However, regulatory requirements for such animal based products may be dynamic and can change from time to time and vary from country to country. Additionally, some countries do not permit any animal-sourced product for human administration. Moreover, due to additional regulatory requirements, the regulatory approval process may be more complex and take longer for products that include an animal sourced product as compared to those products that do not include animal sourced products. Synthetic sealants made from synthetic polymers, or plant-based products are not, however, subjected to such stringent regulatory issues, and may be advantageous and desirable in some applications.

### BRIEF SUMMARY OF THE DISCLOSURE

Disclosed herein is a method for sealing a vascular graft. The method includes applying a hydrogel sealant to the vascular graft thereby sealing the vascular graft. The hydrogel sealant includes a human or plant sourced material and a crosslinking agent.

Also disclosed herein is a vascular graft including a biocompatible material and a crosslinked hydrogel sealant. The crosslinked hydrogel sealant seals the vascular graft such that the vascular graft has a water seepage rate less than about 0.5 mL/min/cm².

Also disclosed herein is a method for sealing a vascular graft. The method includes applying a hydrogel sealant to the vascular graft thereby sealing the vascular graft. The hydrogel sealant includes an activated poly(ethylene) oxide and a poly(amine) and seals the vascular graft such that the vascular graft has a water seepage rate less than about 0.5 mL/min/cm².

### BRIEF DESCRIPTION OF THE DRAWINGS

FIG. 1 illustrates one example of a Left Ventricular Assist Device (LVAD) including an outflow vascular graft.

FIG. 2 illustrates one example of a Paracorporeal Ventricular Assist Device (PVAD) including both an LVAD and a right ventricular assist device (RVAD) each including inflow and outflow vascular grafts.

FIG. 3 illustrates the inside and outside surfaces of a vascular graft after application of a hydrogel sealant comprising SS-PEO-SS and recombinant human serum albumin at different magnifications in accordance with the present disclosure.

FIG. 4 illustrates the inside and outside surfaces of a vascular graft after application of a hydrogel sealant comprising PEO and a tri-(Lys) crosslinking agent at different magnifications in accordance with the present disclosure.

FIG. 5 illustrates a valve conduit for a heart valve replacement that includes an inflow conduit including a hydrogel sealant.

Corresponding reference characters indicate corresponding parts throughout the several views of the drawings. The drawings are not necessarily to scale.

### DETAILED DESCRIPTION OF THE DISCLOSURE

Ventricular assist devices (VADs) are used to provide circulatory support to a patient on either a short term or long term basis depending on the nature of the coronary deficiency. The VAD takes blood from a lower chamber of the heart and helps pump it to the body and vital organs of the patient in the same manner as the heart. There are two basic types of VADs: a left ventricular assist device (LVAD) and a right ventricular assist device (RVAD).

The LVAD is the most common type of VAD. It assists the left ventricle in pumping blood to the aorta. The aorta is the main artery that carries oxygen-rich blood from the heart to the body. For some patients, another acceptable nearby artery is connected to the LVAD instead. In some instances, the LVAD is attached to the bottom of the heart of the patient and requires a vascular graft to transport blood from the LVAD to the circulatory system of the patient (an outflow graft) or to transport blood from the ventricle of the heart to the pump (an inflow graft). In some embodiments described herein, the vascular graft is an outflow graft for an LVAD. In other embodiments described herein, the vascular graft is an inflow graft for an LVAD. In other embodiments, vascular grafts as disclosed herein may also be used with RVADs, which can be used for short or long term support. An RVAD helps the right ventricle pump blood to the pulmonary artery for reoxygenation in the lungs.

The present disclosure provides methodologies for utilizing a hydrogel sealant material to provide sealing characteristics to large-diameter knitted, woven, or other polyester vascular grafts and the like to seal the graft to make it substantially impermeable to blood upon use in the human body. The present disclosure also provides sealed vascular grafts including a hydrogel sealant material. The hydrogel sealants as described herein desirably degrade over a desired time period in vivo as further discussed herein. Although discussed primarily herein in combination with vascular grafts used with ventricular assist medical devices and heart replacement valves, it will be understood by one of skill in the art based on the disclosure herein that the sealing methodologies using the hydrogel sealants and vascular grafts including the hydrogel sealant described herein may be equally applicable to other vascular grafts and medical devices as well.

As used herein, the term “animal” specifically excludes humans. An animal sourced product may include material from any number of animals including, but not limited to, bovine, porcine, canine, and ovine. One specific example of an animal sourced product relevant to this disclosure is bovine serum albumin (BSA). While commonly used in many biological applications, BSA is derived from an animal sourced material. As such, BSA is specifically excluded from use with the methods and compositions herein. In contrast, recombinant products using genes derived from non-animal sources, such as plant or human sources, are specifically included for the methods and compositions herein. In some embodiments, the recombinant products are entirely plant (e.g., rice or yeast) sourced material. In other embodiments the recombinant products are entirely human sourced material. In still other embodiments, the recombinant products are a combination of plant and human sourced material. Additionally, synthetic materials, such as, for example, biocompatible polymers, are suitable and may be included for use for the methods and compositions herein. Synthetic materials can be combined with plant sourced material, human source material, or both in some embodiments.

A gel is a substance with properties intermediate between the liquid and solid states. Gels deform elastically and recover, yet will often flow at higher stresses. They have extended three-dimensional network structures and are highly porous. Accordingly, many gels contain a very high proportion of liquid to solid. The network structures can be permanent or temporary and are based on polymeric molecules. Thus, a hydrogel may be described as a gel, the liquid constituent of which is water.

As used herein, the term “hydrogel” means a polymeric material that swells in water without dissolving and retains a significant amount of water in its structure. Such a material has properties intermediate between liquid and solid states. Thus, for purposes of this disclosure hydrogels are water-swollen, three-dimensional networks of hydrophilic polymers. The polymers may be naturally occurring, synthetic, or a combination thereof.

As used herein, the terms “seal” and “sealing” mean to reduce the porosity of a material, such as a knitted or woven graft material as described herein. Reducing the porosity of a material is not intended to mean a reduction in the porosity of a material to zero in all embodiments, although a reduction in the porosity of a material to zero or substantially zero is within the scope of the embodiments of the present disclosure.

As noted, vascular grafts are commonly used when a blood vessel in a patient needs to be either replaced or rerouted. In some instances, a vascular graft can use a blood vessel from elsewhere in either the same patient or from a donor. As noted, a vascular graft may be used on either a short term or long term basis, depending on the type of device being utilized. In some instances, the vascular graft is synthetic. Synthetic vascular grafts are generally formed from many different biocompatible materials including, but not limited to, poly(ethylene)terephthalate, PTFE, ePTFE, poly(ester), poly(urethane), poly(caprolactone), poly(dioxanone), poly(glycerol sebacate), cellulose, and combinations thereof. In other desirable embodiments, the vascular graft comprises poly(ethylene)terephthalate. In some embodiments within the scope of the present disclosure, the vascular graft comprises two or more different polymers that may or may not be woven or knitted together.

The hydrogel sealants of the present disclosure as described herein for use in combination with the vascular grafts are desirably biocompatible and safely, substantially or completely, break down and/or dissolve in vivo as noted above. In some aspects the hydrogel sealant degrades in vivo in less than 100 days, less than 90 days, less than 80 days, less than 70 days, less than 60 days, less than 50 days, less than 40 days, less than 30 days, less than 20 days, less than 15 days, or less than 10 days. In some aspects, the sealant degrades in from five to 100 days, in from 10 to 60 days, or in from 15 to 45 days. One skilled in the art will recognize based on the disclosure herein that the biocompatible grafts can be configured to have a desired degradation time cycle or cycles depending upon a desired application.

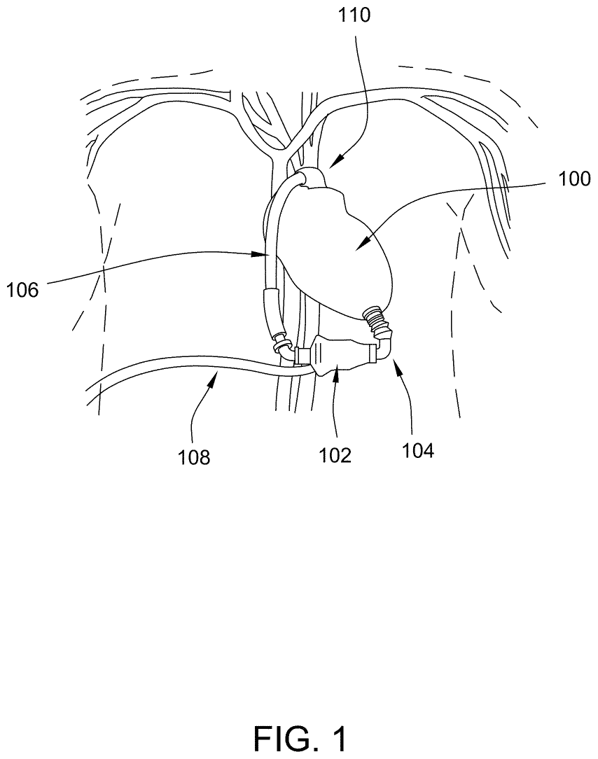

FIG. 1 shows one example of an LVAD 102 including an outflow vascular graft 106 and an inflow vascular graft (or conduit) 104. Heart 100 includes an LVAD 102 having inflow conduit vascular graft 104 and outflow vascular graft 106 that connects to aorta 110. Also shown is a driveline 108 (also called a control cable) that leads to an LVAD controller (not shown in FIG. 1).

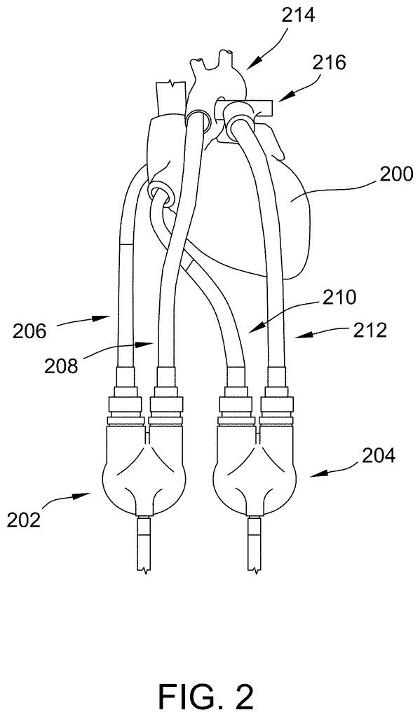

With reference to FIG. 2, heart 200 of a patient includes both an LVAD 202 and an RVAD 204 that can be either a paracorporeal or an implantable device. Both LVAD 202 and RVAD 204 have an inflow vascular graft or conduit 206 (LVAD) and 210 (RVAD), and an outflow vascular graft or conduit 208 (LVAD) and 212 (RVAD). Inflow grafts 206 and 210 connect heart 200 directly to either LVAD 202 or RVAD 204 while outflow grafts 208 and 212 connect the device to either the aorta 214 or pulmonary artery 216. In some instances (not shown in FIG. 2), outflow graft 208 may connect to an artery other than aorta 214. Other configurations for an LVAD and/or an RVAD with vascular grafts are also within the scope of the present disclosure.

In other embodiments of the present disclosure, the vascular graft as described herein may be used for an aortic root replacement in order to repair the lower portion of the aorta of a patient where it connects to the heart. Aortic root replacement can be done either with or without aortic valve replacement. In some aspects, the vascular graft is a value conduit used for heart valve replacement.

Due to the required flexibility of most vascular grafts used inside of the human body, such grafts are frequently formed from a tight mesh, weave, or knit that is not, by itself, impermeable to fluids. Fluid in this context refers to water, blood and other body fluids that may generally come into contact with the vascular graft inside of the human body. In order to reduce the amount of fluid seepage through the walls of the graft and improve performance of the graft upon use in the human body, a biocompatible hydrogel sealant may be applied to the graft in accordance with the present disclosure. The hydrogel sealant may be applied as a single layer, or as multiple layers depending upon the intended application. The hydrogel sealant may be applied to the entire length of the graft, or to only a portion of the length of the graft, and may be applied over one or more surfaces of the graft. Over time once inside the body, tissue grows and forms around the vascular graft as the hydrogel sealant biodegrades, thereby preventing or reducing any later seepage. In some aspects, when the water seepage rate is measured at 200 mmHg of pressure, the seepage rate is reduced to less than about 2.0 mL/min/cm², to less than about 1.5 mL/min/cm², to less than about 1.25 mL/min/cm², to less than about 1.0 mL/min/cm², to less than about 0.75 mL/min/cm², to less than about 0.5 mL/min/cm², or to less than about 0.25 mL/min/cm². In some aspects, the seepage rate is reduced to zero, meaning the sealant makes the vascular graft impermeable to liquids when measured less than 200 mmHg of pressure. Such a seepage rate of zero may be desirable in many embodiments.

As noted, when applied to a vascular graft as described herein, a desired hydrogel may serve as a sealant, thereby reducing or eliminating the blood seepage from the graft. One example of a suitable hydrogel sealant is prepared from human serum albumin and a suitable crosslinking agent. Other non-animal sources of albumin, such as, but not limited to, plant sources (e.g., rice or yeast), are also acceptable for use in the preparation of the hydrogel sealants described herein. The albumin can be prepared by isolation from a natural source or prepared using recombinant DNA techniques. In some embodiments, the albumin is a plant derived recombinant human serum albumin.

Many crosslinking agents for albumin may be suitable for use with the hydrogels herein. One non-limiting example of a crosslinking agent is end group activated poly(ethylene)oxide (PEO) (also known as poly(ethylene)glycol (PEG)). The crosslinking agent may be straight chain or branched and have a number average molecular weight of from 1 kD to 10 kD. In some aspects, the number average molecular weight of the PEO crosslinking agent is about 1 kD, about 2 kD, about 3 kD, about 4 kD, about 5 kD, about 6 kD, about 7 kD, about 8 kD, about 9 kD, or about 10 kD. About as used in this context means ±0.5 kD.

In order to react with the albumin, the crosslinking agent is activated in such a way that functional groups at each end of the crosslinker will react with at least one functional group on the albumin. In some embodiments, the crosslinking agent comprises at least one reactive ester selected from the group consisting of succinimidyl succinate, succinimidyl valerate, succinimidyl propionate, succinimidyl glutarate, succinimidyl carbonate, succinimidyl amido succinate, and combinations thereof. In some embodiments the reactive ester is succinimidyl succinate. In other embodiments the reactive ester is succinimidyl valerate. In still other embodiments, the reactive ester is succinimidyl propionate. In some other embodiments the reactive ester is succinimidyl glutarate. In other embodiments the reactive ester is succinimidyl carbonate. In some embodiments the reactive ester is succinimidyl amido succinate. In some aspects, two different reactive esters are present on the crosslinker.

Another non-limiting example of a synthetic hydrogel sealant suitable for use with the various methodologies described herein includes the combination of a PEO and a poly(amine). In some embodiments, the PEO is a multi-arm star polymer having from 3 to 8 arms, 3 arms, 4 arms, 5 arms, 6 arms, 7 arms, and 8 arms. Each arm, independently of the other arms present, has a number average molecular weight of from 1 kD to 10 kD. In some embodiments, each arm has a number average molecular weight of about 1 kD or 2 kD, or 3 kD, or 4 kD, or 5 kD, or 6 kD, or 7 kD, or 8 kD, or 9 kD, or even 10 kD. About as used in this context means±0.5 kD.

The PEO may be activated as described above for PEO crosslinking agents. Because the PEO includes an activated ester, it will react with an amine. A poly(amine) then serves as a crosslinking agent. In some embodiments, the poly(amine) comprises from 2 to 10 amine functional groups, including 3 amine functional groups, or 4 amine functional groups, or 5 amine functional groups, or 6 amine functional groups, or 7 amine functional groups, or 8 amine functional groups, or 9 amine functional groups or even 10 amine functional groups. In some embodiments, the amines in the poly(amine) are primary amines, secondary amines, or a combination thereof. Poly(peptides) have amine functional groups both on one terminus and on some sidechains, and thus are one non-limiting example of a poly(amine) suitable for use herein. In some embodiments, the poly(amine) is a poly(peptide) comprising α-amino acids and β-amino acids where each amino acid is selected independently of any other amino acid. Each amino acid may be D or L independent of any other amino acid. In other embodiments, the poly(amine) comprises at least one lysine amino acid. In still other embodiments, the poly(amine) is a Lys-Lys-Lys tripeptide.

Also disclosed herein are related methods for reducing permeability in a vascular graft. The methods generally include applying a biocompatible hydrogel sealant for the vascular graft to an exterior or other surface of the vascular graft, wherein the sealant is configured to reduce the permeability of the vascular graft. In some embodiments, the sealant is a biocompatible hydrogel as disclosed elsewhere herein.

Further disclosed herein are related methods for coating a vascular graft with a hydrogel sealant to impart one or more beneficial properties to the vascular graft. The method generally includes preparing a first solution; preparing a second solution; and applying the first solution and the second solution simultaneously to the vascular graft such that the first solution and the second solution mix during the applying thereby coating the vascular graft with a hydrogel sealant; and wherein mixing the first solution and the second solution causes the formation of the hydrogel sealant on the vascular graft.

Further disclosed herein are related methods for coating a vascular graft with a hydrogel sealant to impart one or more beneficial properties to the vascular graft. The method generally includes preparing a first solution; preparing a second solution; applying the first solution to a surface of the vascular graft followed by applying the second solution to the vascular graft such that the first solution and the second solution mix when the second solution contacts the first solution on the surface of the vascular graft, thereby coating the vascular graft with a hydrogel sealant; and wherein mixing the first solution and the second solution causes the formation of the hydrogel sealant on the vascular graft.

Applying the first solution and applying the second solution to the vascular graft can be done simultaneously or sequentially, as noted. In one non-limiting example, a dual chamber syringe is used to hold each solution separately. Upon application, the two solutions combine and mix in the tip of the syringe such that they exit the syringe tip simultaneously as a mixture. In one alternative non-limiting example, the first solution is applied to the surface of the vascular graft, and the second solution is applied thereafter. The time period between applying the first solution and the second solution is such that the first solution does not degrade, evaporate or otherwise become ineffective. Upon applying the second solution to the surface of the vascular graft that comprises the first solution, the two solutions mix and hydrogel formation begins.

By way of example and not limitation, the first solution may include albumin and a second solution may include a crosslinking agent, such as an activated PEO. In yet another non-limiting example, the first solution may include an activated PEO and the second solution includes a poly(amine). Two non-limiting examples of a hydrogel sealant suitable for use with this method are the albumin/PEO and PEO/poly(amine) hydrogels disclosed elsewhere herein. Two or more hydrogel materials or systems may also be used in combination in some embodiments to provide a desired sealing function.

In some embodiments, the methods further include placing the vascular graft on a mandrel before applying the first solution and the second solution. The mandrel may be spun to evenly coat the vascular graft. Spinning the graft is done at a rate such that the hydrogel forms evenly over the vascular graft but not too fast such that centrifugal force causes the hydrogel to dislodge from the vascular graft.

In some embodiments, the hydrogel on the vascular graft is dehydrated after formation for further improve performance of the vascular graft. Dehydration can be done using methods known in the art. One non-limiting example of a dehydrating method includes placing the vascular graft coated with the hydrogel sealant in a non-aqueous solution thereby exchanging the water in the hydrogel for another solvent. In some embodiments, an alcohol and glycerin solution is used to dehydrate the hydrogel sealant on the vascular graft. Alcohols such as methanol, ethanol, propanol, and isopropanol are particular suitable for dehydrating the hydrogel. In some embodiments the alcohol is methanol, ethanol, propanol, isopropanol, or combinations thereof.

In other embodiments, dehydrating the hydrogel includes replacing the water in the hydrogel with glycerin. This is done by placing the vascular graft coated with the hydrogel sealant in a non-aqueous solution thereby exchanging the water for the glycerin. In some aspects, the non-aqueous solution is a mixture of two or more solvents. In some aspects, the non-aqueous solution is a mixture of glycerin and an alcohol as described elsewhere herein. In one non-limiting example, a mixture of glycerin and an alcohol is used. The non-aqueous solution comprises from about 10% to about 90% of glycerin. In some aspects, the non-aqueous solution comprises about 10%, about 15%, about 20%, about 25%, about 30%, about 35%, about 40%, about 45%, about 50%, about 55%, about 60%, about 65%, about 70%, about 75%, about 80%, about 85%, or about 90% glycerin. In some aspects, the non-aqueous solution comprises about 10%, about 15%, about 20%, about 25%, about 30%, about 35%, about 40%, about 45%, about 50%, about 55%, about 60%, about 65%, about 70%, about 75%, about 80%, about 85%, or about 90% alcohol. The alcohol is as described elsewhere herein.

Further, in some embodiments, the method also includes removing the alcohol in the dehydrated hydrogel sealant after dehydrating. Alcohol removal is done using methods known in the art such as, for example, under reduced pressure, increased temperature, or a combination thereof.

In some optional embodiments, the methods described herein further include sterilizing and/or packaging of the vascular graft coated with the hydrogel sealant. In some aspects, the vascular graft coated with the hydrogel sealant is both sterilized and packaged for storage and later use. In some aspects, the vascular graft coated with the hydrogel sealant is sterilized and used inserted into a patient immediately thereafter.

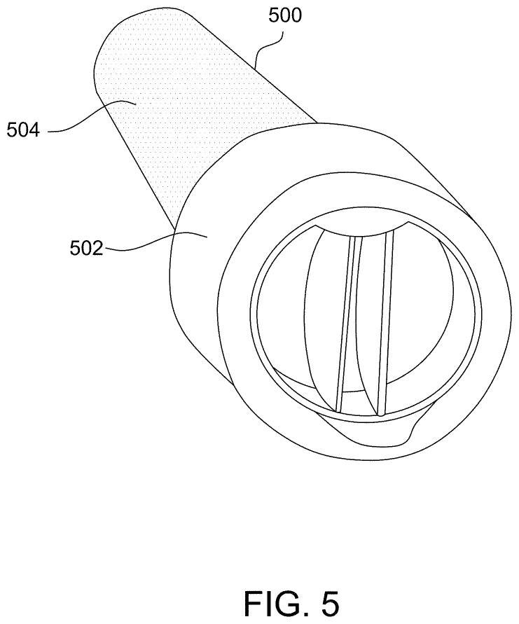

In a further non-limiting embodiment of the present disclosure, a vascular graft as disclosed herein may be attached to a heart replacement valve (or similar type of valve) for placement inside of a patient. Prior to the introduction of the replacement heart valve/vascular graft combination into the patient, the vascular graft may be sealed with a hydrogel sealant as described elsewhere herein. FIG. 5 shows a heart valve replacement 502 attached to inflow conduit vascular graft 500. Inflow conduit vascular graft 500 includes hydrogel sealant 504 on a surface thereof to impart a desired sealing function as described herein.

### Example 1

In Example 1, an albumin based hydrogel sealant is prepared and applied to a specific graft material and the resulting water permeability of the hydrogel sealed graft material determined.

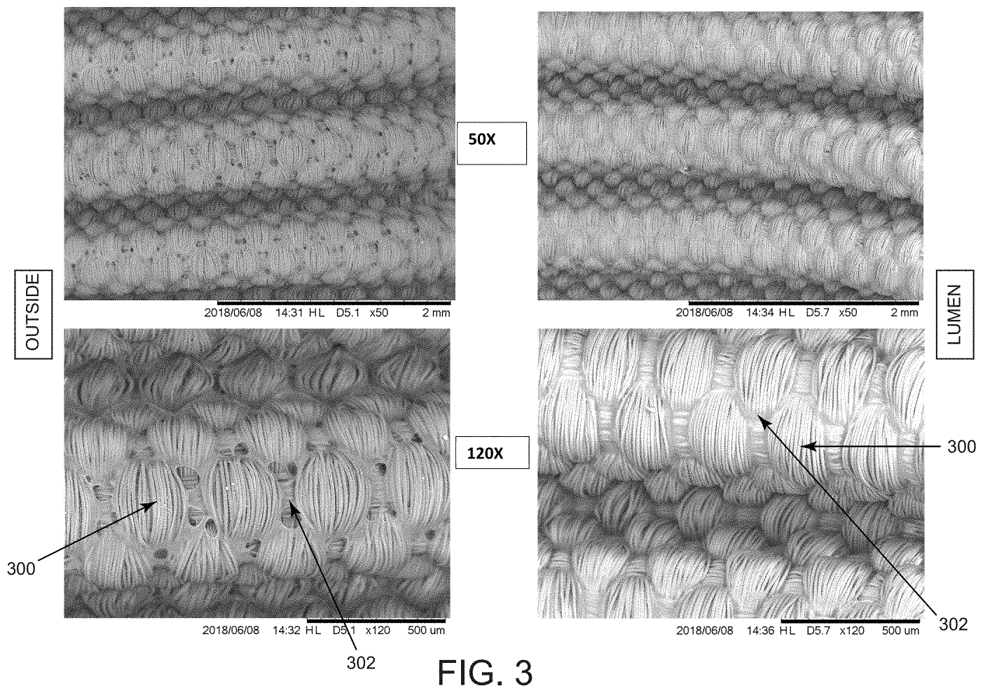

A 30% solution of rHSA was dissolved in a pH 9.6 carbonate-bicarbonate solution, and s bifunctional activated ester SS-PEO-SS (molecular weight 3,400 g/mole) was dissolved in a pH 7.4 phosphate buffer solution at a ratio of 0.130 g of SS-PEO-SS per mL of the albumin solution. The two solutions were loaded in two separate syringes, and the vascular graft (8 mm to 22 mm) was stretched over a mandrel and mounted on a motor. The two solutions were sprayed onto the graft while it was axially spinning at about 600 rpm. The reaction between the rHSA and PEO takes place within a few seconds to form the hydrogel sealant (FIG. 3). As shown in FIG. 3, hydrogel sealant 302 is closely intertwined with fabric 300 both on the exterior and in the lumen of the vascular graft. This intertwining reduces the permeability of the vascular graft.

The water in the hydrogel was removed and stabilized by dehydrating the hydrogel in an isopropanol/glycerin (75/25) mixture overnight. The water in the hydrogel was replaced with glycerin to prevent hydrolysis of the hydrogel. A 14 mm PET low porosity graft thus coated was tested for water permeability at 200 mmHg pressure. The graft was filled with water and subjected to 200 mmHg pressure and water permeability measured. The water permeability was 1.2 ml/min/cm².

### Example 2

In Example 2, an 4-ARM PEG with Lys-Lys-Lys based hydrogel sealant is prepared and applied to a specific graft material and the resulting water permeability of the hydrogel sealed graft material determined.

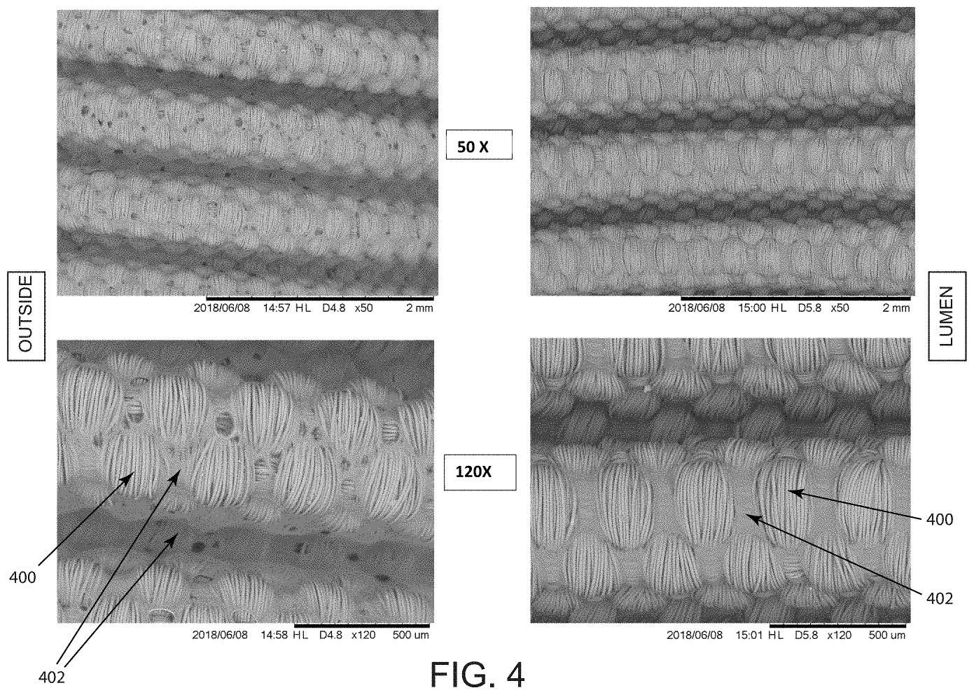

A 4-ARM-PEO-SG (0.675 g; 6.2 μmol) was dissolved in 4 mL of PBS (pH 7.4), and Lys-Lys-Lys (0.025 g; 6.2 μmol) was dissolved in 4 mL of carbonate-bicarbonate buffer (pH 9.6), separately. The two solutions were loaded on a dual syringe cartridge separately. A 14 mm low porosity graft was stretched on a mandrel and the mandrel was attached to a motor. The graft was spun axially at approximately 600 rpm. The solution was mixed and sprayed on to the rotating graft under nitrogen pressure to form a uniform coating on the graft. The hydrogel was allowed to setup over a period of about 15 min. The water in the hydrogel was removed and stabilized by dehydrating the hydrogel in isopropanol/glycerin (75/25) mixture overnight. The water in the hydrogel was replaced with glycerin to prevent hydrolysis of the hydrogel (FIG. 4). As shown in FIG. 4, hydrogel sealant 402 is closely intertwined with fabric 400 both on the exterior and in the lumen of the vascular graft. This intertwining reduces the permeability of the vascular graft.

The 14 mm PET low porosity graft thus coated was tested for water permeability at 200 mmHg pressure. The graft was filled with water and subjected to 200 mmHg pressure and water permeability measured. The graft was essentially non-permeable to water at 200 mmHg pressure.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the subject matter herein, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for sealing a vascular graft, the method comprising:
applying a hydrogel sealant to the vascular graft thereby sealing the vascular graft, wherein:
the hydrogel sealant includes a human or plant sourced material and a crosslinking agent.

2. The method of claim 1, wherein the hydrogel sealant seals the vascular graft such that the vascular graft has a water seepage rate less than about 0.5 mL/min/cm².

3. The method of claim 1, wherein the hydrogel sealant is applied to the vascular graft by applying a first solution including the human or plant sourced material and a second solution including the crosslinking agent to the vascular graft, wherein the first solution and the second solution are applied simultaneously or sequentially.

4. The method of claim 3, wherein the vascular graft is placed on a mandrel prior to application of the first solution and the second solution.

5. The method of claim 1, wherein the vascular graft is dehydrated after application of the hydrogel sealant.

6. The method of claim 1, wherein the human or plant sourced material comprises albumin and the crosslinking agent comprises an activated poly(ethylene)oxide.

7. A vascular graft comprising a biocompatible material and a crosslinked hydrogel sealant.

8. The vascular graft of claim 7, wherein the crosslinked hydrogel sealant seals the vascular graft such that the vascular graft has a water seepage rate less than about 0.5 mL/min/cm².

9. The vascular graft of claim 7, wherein the biocompatible material is selected from the group consisting of poly(ethylene)terephthalate, PTFE, ePTFE, poly(ester), poly(urethane), poly(caprolactone), poly(dioxanone), poly(glycerol sebacate), cellulose, and combinations thereof.

10. The vascular graft of claim 7, wherein the crosslinked hydrogel sealant comprises an activated poly(ethylene) oxide and a poly(amine).

11. The vascular graft of claim 10, wherein the poly(ethylene) oxide is a multi-arm star polymer.

12. The vascular graft of claim 10, wherein the poly(amine) includes from 2 to 10 amine functional groups.

13. The vascular graft of claim 7, wherein the crosslinked hydrogel sealant comprises a human or plant sourced material and a crosslinking agent.

14. The vascular graft of claim 13, wherein the human or plant sourced material comprises albumin and the crosslinking agent comprises an activated poly(ethylene)oxide.

15. The vascular graft of claim 14, wherein the activated poly(ethylene)oxide has a number average molecular weight of from 1 kD to 10 kD.

16. The vascular graft of claim 14, wherein the activated poly(ethylene)oxide comprises at least one reactive ester selected from the group consisting of succinimidyl succinate, succinimidyl valerate, succinimidyl propionate, succinimidyl glutarate, succinimidyl carbonate, succinimidyl amido succinate and combinations thereof.

17. A method for sealing a vascular graft, the method comprising:
applying a hydrogel sealant to the vascular graft thereby sealing the vascular graft, wherein:
the hydrogel sealant includes an activated poly(ethylene) oxide and a poly(amine), and wherein
the hydrogel sealant seals the vascular graft such that the vascular graft has a water seepage rate less than about 0.5 mL/min/cm².

18. The method of claim 17, wherein the hydrogel sealant is applied to the vascular graft by applying a first solution including the activated poly(ethylene) oxide and a second solution including the poly(amine) to the vascular graft, wherein the first solution and the second solution are applied simultaneously or sequentially.

19. The method of claim 18, wherein the vascular graft is placed on a mandrel prior to the applying of the first solution and the second solution.

20. The method of claim 17, wherein the vascular graft is dehydrated after application of the hydrogel sealant.
